(19) United States
(12) Patent Application Publication
(10) Pub. No.: US 2013/0208243 A1
(43) Pub. Date: Aug. 15, 2013

(54) OPHTHALMOLOGIC APPARATUS, METHOD FOR CONTROLLING OPHTHALMOLOGIC APPARATUS, AND STORAGE MEDIUM

(71) Applicant: CANON KABUSHIKI KAISHA, (US)

(72) Inventor: Wataru Sakagawa, Kawasaki-shi (JP)

(73) Assignee: CANON KABUSHIKI KAISHA, Tokyo (JP)

(21) Appl. No.: 13/765,491

(22) Filed: Feb. 12, 2013

(30) Foreign Application Priority Data
Feb. 15, 2012 (JP) 2012-030665

(51) Int. Cl. A61B 3/00

(52) U.S. Cl. CPC A61B 3/0091
USPC 351/211; 351/246

(57) ABSTRACT

An ophthalmologic apparatus includes a first changing unit configured to change a size of an aperture of a diaphragm arranged in an optical path connecting a subject's eye and a light source and in a position conjugate with a pupil of the subject's eye, and a second changing unit configured to, if a signal for instructing the first changing unit to change the size of the aperture from a first size to a second size smaller than the first size is output to the first changing unit, change an amount of light of a fixation target image projected onto the subject's eye from a first light amount to a second light amount smaller than the first light amount.

### BACKGROUND OF THE INVENTION

1. Field of the Invention

The present invention relates to an ophthalmologic apparatus, a method for controlling the ophthalmologic apparatus, and a storage medium.

2. Description of the Related Art

An ophthalmologic apparatus, such as an eye refractive power measurement apparatus and an ophthalmologic imaging apparatus, conventionally includes an optical system for projecting a fixation target onto a subject's eye. The eye refractive power measurement apparatus uses the fixation target to promote relaxation of the subject's eye. The ophthalmologic imaging apparatus uses the fixation target to fixate the subject's eye. Depending on the brightness of the fixation target, the subject's eye produces miosis, in which case desired measurement and test results may fail to be obtained due to partial shielding by the iris of the subject's eye. In particular, an eye having a small pupil diameter is known to have a tendency to perceive the fixation target as glaring and easily produce miosis.

Japanese Patent Application Laid-Open No. 06-189904 discusses an eye refractive power measurement apparatus that is configured to dim out the fixation target for an eye having a small pupil diameter. Japanese Patent No. 4233426 discusses reducing a diaphragm diameter if a ring light flux is shielded by the iris of the subject's eye.

The operations for reducing the diaphragm diameter and dimming the fixation target for an eye having a small pupil diameter are troublesome to the examiner. Miosis may develop further if it takes longer to dim the fixation target after the determination of a small pupil because of the operation troublesomeness. There has thus been a problem that appropriate measurement and test results may fail to be obtained.

### SUMMARY OF THE INVENTION

The present invention is directed to an ophthalmologic apparatus capable of promptly obtaining appropriate measurement and test results about an eye having a small pupil diameter without a troublesome operation.

The present invention is also directed to providing operations and effects that are derived from configurations described in exemplary embodiments of the present invention to be described below but not obtainable by conventional techniques.

According to an aspect of the present invention, an ophthalmologic apparatus includes a first changing unit configured to change a size of an aperture of a diaphragm arranged in an optical path connecting a subject's eye and a light source and in a position conjugate with a pupil of the subject's eye, and a second changing unit configured to, if a signal for instructing the first changing unit to change the size of the aperture from a first size to a second size smaller than the first size is output to the first changing unit, change an amount of light of a fixation target image projected onto the subject's eye from a first light amount to a second light amount smaller than the first light amount.

According to exemplary embodiments of the present invention, appropriate measurement and test results about an eye having a small pupil diameter may be promptly obtained without a troublesome operation.

Further features and aspects of the present invention will become apparent from the following detailed description of exemplary embodiments with reference to the attached drawings.

### BRIEF DESCRIPTION OF THE DRAWINGS

The accompanying drawings, which are incorporated in and constitute a part of the specification, illustrate exemplary embodiments, features, and aspects of the invention and, together with the description, serve to explain the principles of the invention.

FIG. 1 is a layout diagram illustrating optical systems in a measurement section of an eye refractive power measurement apparatus according to a first exemplary embodiment of the present invention.

FIG. 2 is an external view of the eye refractive power measurement apparatus according to the first exemplary embodiment.

FIG. 3 is a comparison diagram illustrating two types of eye refractive power measurement diaphragms according to the first exemplary embodiment.

FIG. 4 is a system block diagram of the eye refractive power measurement apparatus according to the first exemplary embodiment.

FIGS. 5A, 5B, and 5C are flowcharts illustrating examples of the operation of the eye refractive power measurement apparatus according to the first exemplary embodiment.

FIG. 6 is a flowchart illustrating detailed control when an amount of light of a fixation target is maintained or changed to a light amount smaller than that for a standard eye in advance regardless of a size of a diaphragm aperture.

FIG. 7 is a layout diagram illustrating optical systems in an ophthalmologic imaging apparatus according to a second exemplary embodiment.

FIG. 8 is a front view of a switchable crystalline lens baffle according to the second exemplary embodiment.

FIGS. 9A, 9B, and 9C are flowcharts illustrating examples of the operation of the ophthalmologic imaging apparatus according to the second exemplary embodiment.

### DESCRIPTION OF THE EMBODIMENTS

Various exemplary embodiments, features, and aspects of the invention will be described in detail below with reference to the drawings.

### Overall Configuration of Dioptometer

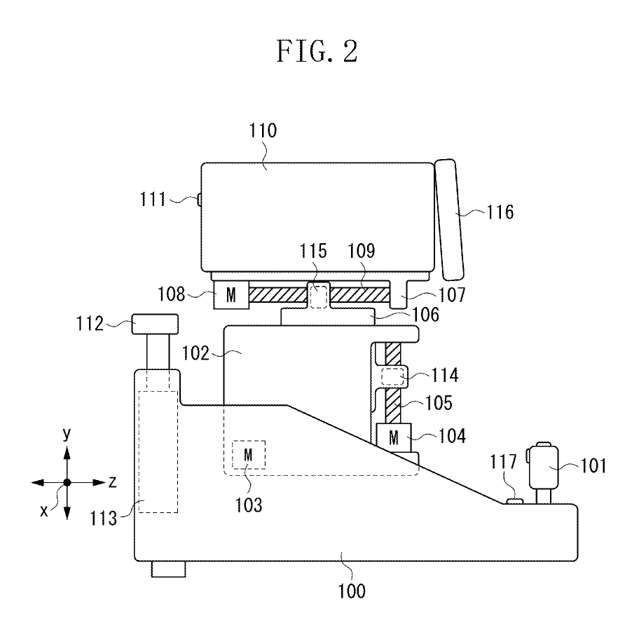

A first exemplary embodiment will be described below. FIG. 2 illustrates a schematic overall configuration diagram of a dioptometer according to the present exemplary embodiment. A frame 102 is movable in a horizontal direction (hereinafter, referred to as an X-axis direction) with respect to a base 100. An X-axis direction drive mechanism includes an X-axis drive motor 103, a feed screw (not illustrated), and a nut (not illustrated). The X-axis drive motor 103 is fixed to the base 100. The feed screw is coupled to an output shaft of the X-axis drive motor 103. The nut can move over the feed screw in the X-axis direction and is fixed to the frame 102. The X-axis drive motor 103 rotates to move the frame 102 in the X-axis direction via the feed screw and the nut. A frame 106 is movable in a vertical direction (hereinafter, a Y-axis direction) with respect to the frame 102.

A Y-axis direction drive mechanism includes a Y-axis drive motor 104, a feed screw 105, and a nut 114. The Y-axis drive motor 104 is fixed to the frame 102. The feed screw 105 is coupled to an output shaft of the Y-axis drive motor 104. The nut 114 can move over the feed screw 105 in the Y-axis direction and is fixed to the frame 106. The Y-axis drive motor 104 rotates to move the frame 106 in the Y-axis direction via the feed screw 105 and the nut 114.

A frame 107 is movable in a front-back direction (hereinafter, a Z-axis direction) with respect to the frame 106.

A Z-axis direction drive mechanism includes a Z-axis drive motor 108, a feed screw 109, and a nut 115. The Z-axis drive motor 108 is fixed to the frame 107. The feed screw 109 is coupled to an output shaft of the Z-axis drive motor 108. The nut 115 can move over the feed screw 109 in the Z-axis direction and is fixed to the frame 106.

The Z-axis drive motor 108 rotates to move the frame 107 in the Z-axis direction via the feed screw 109 and the nut 115. A measurement unit 110 intended for measurement is fixed to the frame 107. A light source 111 intended for alignment is arranged on a subject-side end of the measurement unit 110. The base 100 includes a joystick 101 for controlling a position of the measurement unit 110 and an eye refractive power measurement diaphragm switching key 117 for switching eye refractive power measurement diaphragms to be described below.

For eye refractive power measurement, the subject can put his/her chin on a chin rest 112 and press his/her forehead against a forehead rest portion of a frame of a face rest (not illustrated) fixed to the base 100 to fix the position of the subject's eye. The chin rest 112 can be adjusted in the Y-axis direction according to the size of the subject's face by using a chin rest drive mechanism 113. A liquid crystal display (LCD) monitor 116, which is a display member for observing the subject's eye E, is arranged on an examiner-side end of the measurement unit 110. The LCD monitor 116 can display measurement results.

### Eye Refractive Power Measurement System

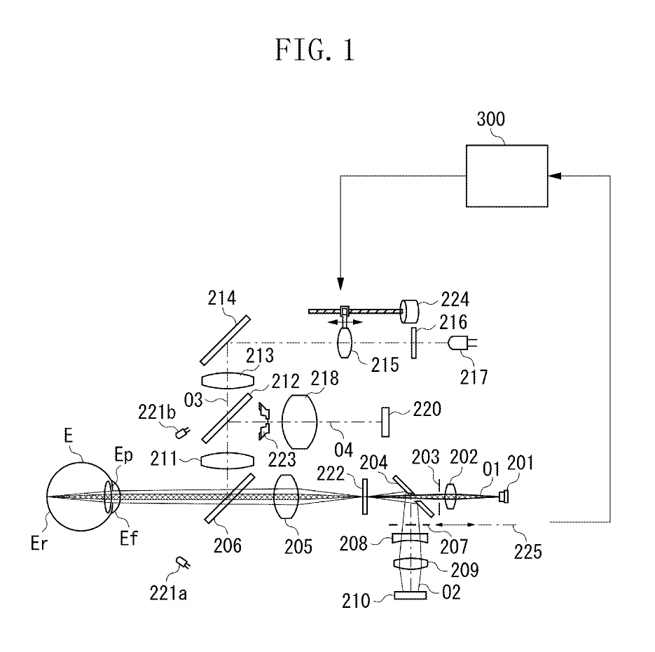

FIG. 1 is a layout diagram illustrating optical systems inside the measurement unit 110. An eye refractive power measurement light source 201 emits light having a wavelength of 880 nm. An optical path 01 extends from the eye refractive power measurement light source 201 to the subject's eye E. A lens 202, a diaphragm 203, a perforated mirror 204, an insertable and removable diffusion plate 222, and a lens 205 are arranged on the optical path 01. The diaphragm 203 is generally conjugate with the pupil Ep of the subject's eye E. A dichroic mirror 206 is further arranged on the optical path 01. The dichroic mirror 206 totally reflects visible light from the side of the subject's eye E and partly reflects the light flux having a wavelength of 880 nm. An eye refractive power measurement diaphragm 207, a light flux spectral prism 208, a lens 209, and an image sensor 210 are arranged in succession on an optical path 02 which extends in a reflecting direction of the perforated mirror 204.

Eye refractive power measurement diaphragms to be positioned generally conjugate with the pupil Ep include a standard pupil diameter diaphragm 207 and a small pupil diameter diaphragm 225. Either one of the diaphragms 207 and 225 is always placed on the optical path 02 by an eye refractive power measurement diaphragm switching solenoid (not illustrated). As employed herein, a standard pupil diameter (a normal pupil diameter) refers to a standard pupil diameter of a standard subject's eye (for example, a pupil diameter greater than 4 mm or greater than 3.3 mm). The standard pupil diameter diaphragm 207 refers to a diaphragm that is suited to the standard pupil diameter of the standard subject's eye.

A small pupil diameter refers to a pupil diameter (i.e., a pupil diameter smaller than 4 mm or smaller than 3.3 mm) smaller than the standard pupil diameter. The small pupil diameter diaphragm 225 refers to a diaphragm that is suited to the small pupil diameter. It should be noted that the standard pupil diameter is not limited to the aforementioned 4 mm or 3.3 mm, and may be other values.

During eye refractive power measurement, the semi-transparent diffusion plate 222 is positioned out of the optical path 01 by a not-illustrated diffusion plate insertion and removal solenoid 510. The eye refractive power measurement light source 201 emits a light flux. The diaphragm 203 narrows the light flux onto the optical path 01. The lens 202 forms a primary image of the light flux in front of the lens 205. The light flux is transmitted through the lens 205 and the dichroic mirror 206 to be projected onto the pupil center of the subject's eye E.

The light flux forms an image on the fundus Er, and the reflected light passes through the pupil center to be made incident on the lens 205 again. The incident light flux is transmitted through the lens 205 and then reflected by the periphery of the perforated mirror 204. The reflected light reflux is pupil-separated by the standard pupil diameter diaphragm 207 or the small pupil diameter diaphragm 225 generally conjugate with the pupil Ep of the subject's eye E. The standard pupil diameter diaphragm 207 and the small pupil diameter diaphragm 225 both have a ring-shaped slit. The pupil-separated light flux is projected onto a light receiving surface of the image sensor 210 as a ring image.

If the subject's eye E is an emmetropic eye, the ring image forms as a predetermined circle. If the subject's eye E is a myopic eye, the ring image forms as a circle smaller than that of an emmetropic eye. If the subject's eye E is a hypermetropic eye, the ring image forms a circle greater than that of an emmetropic eye. If the subject's eye E is astigmatic, the ring image forms an ellipse. The angle formed between the horizontal axis and the major or minor axis of the ellipse is an astigmatic axis angle. Eye refractive power is determined based on the coefficient of the ellipse.

Now, a fixation target projection optical system and an alignment light receiving optical system are arranged in a reflecting direction of the dichroic mirror 206. The alignment light receiving optical system is used both for observation of the anterior segment of the subject's eye E and for alignment detection. A lens 211, a dichroic mirror 212, a lens 213, a folding mirror 214, a lens 215, a fixation target 216, and a fixation target illumination light source 217 are arranged in succession on an optical path 03 of the fixation target projection optical system.

At the time of fixation guiding, the fixation target illumination light source 217 is lit to illuminate the fixation target 216 with a projection light flux from behind. The projection light flux is projected onto the fundus Er of the subject's eye E through the lens 215, the folding mirror 214, the lens 213, the dichroic mirror 212, and the lens 211. In other words, the image of the fixation target 216 is projected onto the subject's eye E. A fixation target light amount control unit 300, which is a function of a system control unit 401 to be described below, can control the amount of the irradiating light from the fixation target illumination light source 217 to control the amount of light of the image of the fixation target 216 projected on the subject's eye E.

To achieve a fogging state of the subject's eye E, the lens 215 is configured to be movable in the direction of the optical axis by a fixation guiding motor 224 which performs diopter guiding control. A display for displaying a fixation target may be used instead of the fixation target 216 and the fixation target illumination light source 217.

An optical path 04 extends in a reflecting direction of the dichroic mirror 212. An alignment prism diaphragm 223, a lens 218, and an image sensor 220 are arranged in succession on the optical path 04. Anterior segment illumination light sources 221a and 221b are arranged near a measurement section of the ophthalmologic apparatus. The anterior segment illumination light sources 221a and 221b are light sources for illuminating the anterior segment of the subject's eye E, and have a wavelength of around 780 nm. A light flux of an anterior segment image of the subject's eye E illuminated by the anterior segment illumination light sources 221a and 221b passes through the optical path 04 to form an image on the image sensor 220.

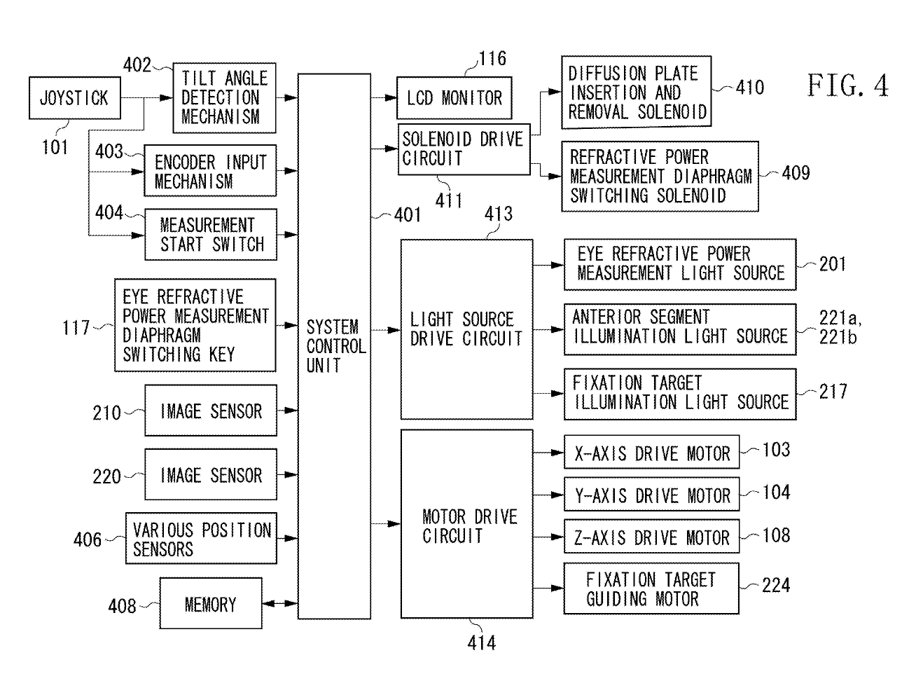

For alignment, the diffusion plate 222 is inserted into the optical path 01 by a diffusion plate insertion and removal solenoid 410 (illustrated in FIG. 4). The foregoing eye refractive power measurement light source 201 is also used as a light source for alignment detection. The diffusion plate 222 is inserted into a position where a primary image of the eye refractive power measurement light source 201 is formed by the projection lens 202. The position coincides with a focal position of the lens 205. Consequently, an image of the eye refractive power measurement light source 201 is formed on the diffusion plate 222 once, and the image serves as a secondary light source to project a wide parallel light flux onto the subject's eye E through the lens 205.

The parallel light flux is reflected by the cornea Ef of the subject's eye E. The reflected light flux is spectrally dispersed through the alignment prism diaphragm 223 and converged on the image sensor 220 through the lens 218. Since the image formed on the image sensor 220 has a luminescent spot in a different position depending on the position of the subject's eye E, the subject's eye E can be aligned based on the position of the luminescent spot.

### Measurement Diaphragm Aperture

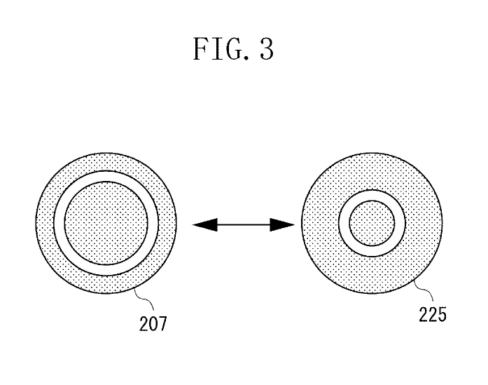

FIG. 3 illustrates the shapes of two types of eye refractive power measurement diaphragm. apertures (ring-shaped openings). The small pupil diameter diaphragm 225 has a ring-shaped slit (a ring-shaped opening) smaller than that of the standard pupil diameter diaphragm 207 in radius (inner and outer diameters). The small pupil diameter diaphragm 225 can thus separate a light flux that passes a closer portion to the cornea center. Portions closer to the cornea center have lower refractive power and are not optimum for eye refractive power measurement. However, such portions are less likely to be shielded by the iris and are suited to the measurement of a subject's eye E having a small pupil diameter.

The small pupil diameter diaphragm 225 may have a ring-shaped opening whose inner diameter alone is smaller than that of the standard pupil diameter diaphragm 207. The small pupil diameter diaphragm 225 and the standard pupil diameter diaphragm 207 correspond to a light flux limiting unit which limits incidence of a light flux on the subject's eye E.

When the examiner operates the eye refractive power measurement diaphragm switching key 117, the standard pupil diameter diaphragm 207 is switched to the small pupil diameter diaphragm 225 if the standard pupil diameter diaphragm 207 has been placed in the optical path 02 before the operation. According to the switching, the fixation target light amount control unit 300 controls the amount of light of the fixation target 216 (the amount of light of the fixation target image projected onto the subject's eye E). In other words, the fixation target light amount control unit 300 maintains or changes the amount of light of the fixation target 216 to a light amount (a second light amount) smaller than a light amount (a first light amount) for the standard eye.

### System Control

FIG. 4 is a system block diagram of the dioptometer according to the present exemplary embodiment. A system control unit 401 controls the entire system. The system control unit 401 includes a program storage unit, a data storage unit, an input/output control unit, and an arithmetic processing unit. The data storage unit stores data for correcting eye refractive power values. The input/output control unit controls various device inputs and outputs. The arithmetic processing unit calculates data obtained from various devices. A control at the start of test, an automatic alignment control, an eye refractive power measurement control, and a fogging control will be described below with reference to FIG. 4.

### 1) Control at the Start of Test

At the start of test, the system control unit 401 initially turns on the eye refractive power measurement light source 201, the anterior segment illumination light sources 221a and 221b, and the fixation target illumination light source 217 via a light source drive circuit 413 to prepare for positioning and eye refractive power measurement. The amount of light of the fixation target illumination light source 217 can be switched between two levels including a normal light amount (a predetermined light amount) corresponding to the standard pupil diameter and a low light amount corresponding to the small pupil diameter. At the start of test, the amount of light of the fixation target illumination light source 217 is set to the normal light amount.

The examiner operates the joystick 101 to position the measurement unit 110 to the subject's eye E. A tilt angle detection mechanism 402, an encoder input mechanism 403, and a measurement start switch 404 are arranged on the joystick 101. The tilt angle detection mechanism 402 is intended to detect a tilt in front, back, right, and left directions. The encoder input mechanism 404 is intended to detect rotation. The measurement start switch 404 is pressed to start measurement.

According to inputs from the tilt angle detection mechanism 402 and the encoder input mechanism 403, the system control unit 401 drives the X-axis drive motor 103, the Y-axis drive motor 104, and the Z-axis drive motor 108 via a motor drive circuit 414 to control the position of the measurement unit 110. At the same time, the system control unit 401 synthesizes an anterior segment image of the subject's eye E captured by the image sensor 220 and character and graphic data, and displays the resultant on the LCD monitor 116.

The examiner observes the anterior segment of the subject's eye E displayed on the LCD monitor 116. If the pupil diameter is determined to be insufficient, the examiner presses the eye refractive power measurement diaphragm switching key 117. The system control unit 401 then operates a refractive power measurement diaphragm switching solenoid 409 to switch between the standard pupil diameter diaphragm 207 and the small pupil diameter diaphragm 225. Since the refractive power measurement diaphragm switching solenoid 409 changes its position depending on which of the standard pupil diameter diaphragm 207 and the small pupil diameter diaphragm 225 is in the optical path 02, the refractive power measurement diaphragm switching solenoid 409 functions as an acquisition unit for acquiring information about the pupil Ep of the subject's eye E by recognizing the position.

### 2) Automatic Alignment Control

When the examiner presses the measurement start switch 404, the system control unit 401 starts the automatic alignment control. In the automatic alignment control, the system control unit 401 analyzes the anterior segment image captured by the image sensor 220 to detect the pupil Ep of the subject's eye E. When the pupil Ep is detected, the system control unit 401 performs X- and Y-axis motor control via the motor drive circuit 414 in directions such that the center axis of the pupil Ep coincides with the optical axis of the measurement unit 110.

When the center axis of the pupil Ep of the subject's eye E generally coincides with the optical axis of the measurement unit 110, reflection of the light from the anterior segment illumination light source 221a and reflection of the light from the anterior segment illumination light source 221b appear on the anterior segment. The system control unit 401 performs X-, Y-, and Z-axis motor control so that the reflections come to a predetermined position and size. The system control unit 401 detects a luminescent spot spectrally dispersed by the alignment prism diaphragm 223, and controls the motor drive circuit 414 according to the position of the luminescent spot. The system control unit 401 then performs X-, Y-, and Z-axis fine motor control. If the position of the luminescent spot falls within a predetermined range, the system control unit 401 completes the automatic alignment control and proceeds to eye refractive power measurement.

### 3) Eye Refractive Power Measurement Control

At the time of eye refractive power measurement, the system control unit 401 retracts the diffusion plate 222, which has been inserted in the optical path 01 for the automatic alignment control, from the optical path 01. The system control unit 401 adjusts the amount of the light from the eye refractive power measurement light source 201 to project a measurement light flux onto the fundus Er of the subject's eye E. Reflected light from the fundus Er travels through the optical path 02 and is received by the image sensor 210. The image sensor 210 captures the reflected light from the fundus Eras a ring-shaped image through the standard pupil diameter diaphragm 207 or the small pupil diameter diaphragm 225. The ring image is stored in a memory 408.

The system control unit 401 calculates the barycentric coordinates of the ring image stored in the memory 408, and determines an ellipse equation by a known method. The system control unit 401 calculates the major and minor diameters of the determined ellipse and the tilt of the major axis to calculate eye refractive power of the subject's eye E, and displays the eye refractive power on the LCD monitor 116. Eye refractive power values corresponding to the major and minor diameters of the determined ellipse and a relationship between the angles of the elliptic axes and the astigmatic axis on the light receiving surface of the image sensor 210 are corrected in advance in a manufacturing process of the ophthalmologic apparatus.

### 4) Fogging Control

In the fogging control, the motor drive circuit 414 drives the lens 215 by using the fixation guiding motor 224, whereby the fixation target image is moved to a position corresponding to an eye refractive power value determined by preliminary measurement. Consequently, the fixation target image is formed on the fundus Er of the subject's eye E. The system control unit 401 then moves the lens 215 further by a predetermined amount to fog the fixation target 216. The fixation target image is formed slightly in front of the fundus Er of the subject's eye E. The subject's eye E is adjusted to be focused on a far side to form a fixation target image.

The adjustment relaxes the subject's eye E. In the present exemplary embodiment, the lens 215 is moved to achieve fogging. In another exemplary embodiment, the lens 215 may be fixed while the fixation target 216 is moved for fogging. The lens 215 and the fixation target 216 both may be moved. The system control unit 401 repeats such a fogging control and the measurement of an eye refractive power value. As a result, an eye refractive power value can be obtained with the subject's eye E sufficiently relaxed. Such is a basic flow of the eye refractive power measurement.

### Subject's Eye Having Small Pupil Diameter

In the present exemplary embodiment, the size of the diaphragm aperture arranged in a position optically conjugate with the pupil Ep of the subject's eye E can be switched between a first size intended for the standard eye and a second size smaller than the first size. If the subject's eye E has a small pupil diameter and the size of the diaphragm aperture has been switched to the second size, the system control unit 401 maintains or changes the amount of light of the fixation target 216 to a light amount smaller than that for the standard eye.

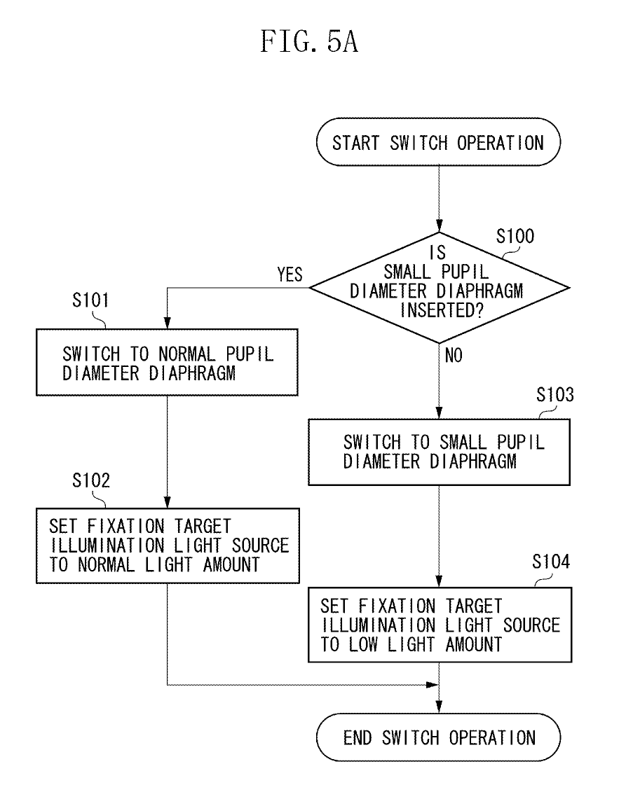

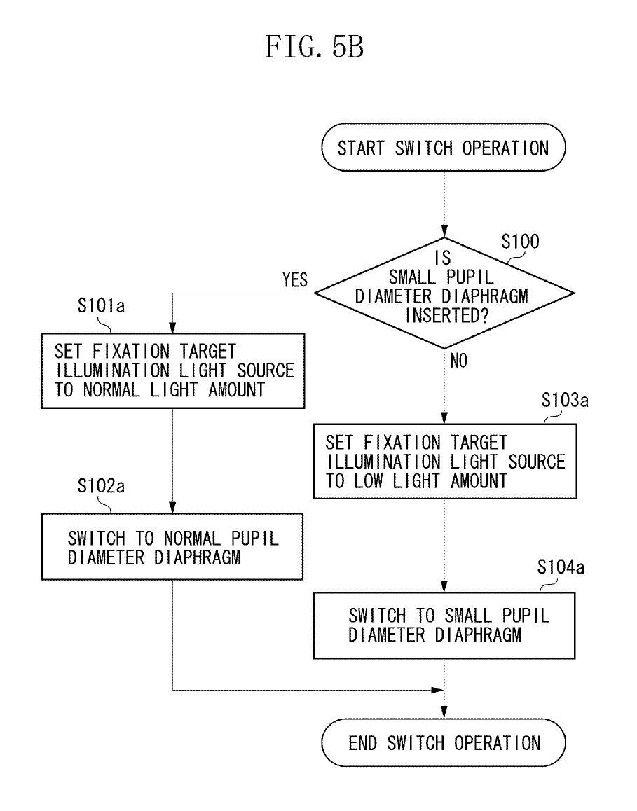

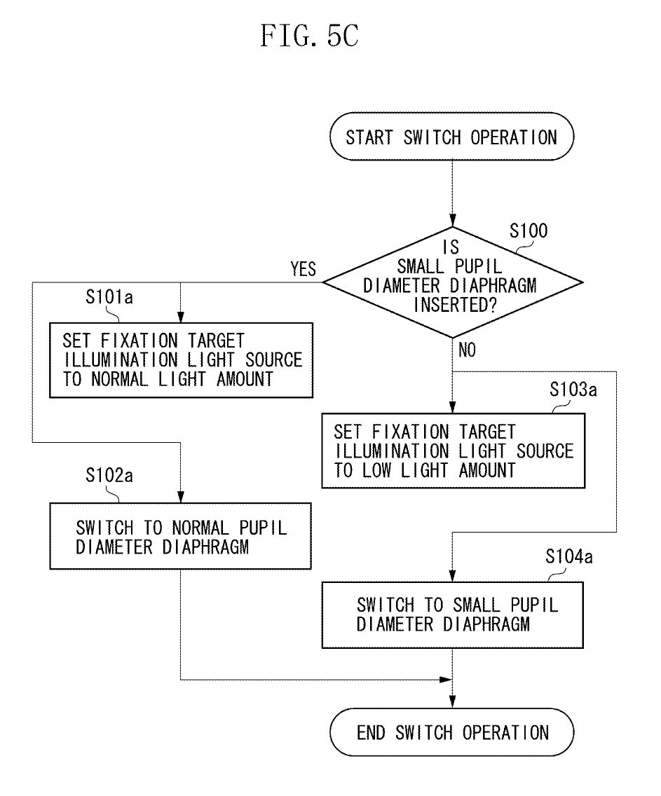

Operations when the eye refractive power measurement diaphragm switching key 117 is pressed will be described in detail below with reference to FIGS. 5A, 5B, and 5C. FIG. 5A is a flowchart when the amount of light of the fixation target 216 is maintained or changed to a light amount smaller than that for the standard eye after switching of the diaphragm aperture. FIG. 5B is a flowchart when the amount of light of the fixation target 216 is maintained or changed to a light amount smaller than that for the standard eye before the switching of the diaphragm aperture. FIG. 5C is a flowchart when the amount of light of the fixation target 216 is maintained or changed to a light amount smaller than that for the standard eye in an interlocked manner with the switching of the diaphragm aperture.

The examiner presses the eye refractive power measurement switching key 117. In step S100, the system control unit 401 determines the eye refractive power measurement diaphragm currently inserted in the optical path 02. In FIG. 5A, if the standard pupil diameter diaphragm 207 is inserted when the examiner presses the eye refractive power measurement switching key 117 (NO in step S100), then instep S103, the system control unit 401 switches to the small pupil diameter diaphragm 225. In step S104, the fixation target light amount control unit 300 sets the fixation target illumination light source 217 to a low light amount.

If the small pupil diameter diaphragm 225 is inserted when the eye refractive power measurement diaphragm switching key 117 is pressed (YES in step S100), then in step S101, the system control unit 401 switches to the standard pupil diameter diaphragm 207 which is a normal pupil diameter diaphragm. In step S102, the fixation target light amount control unit 300 sets the fixation target illumination light source 217 to a normal light amount.

As a result, if the examiner selects an eye refractive power measurement diaphragm suited to the pupil diameter of the subject's eye E, the fixation target illumination light source 217 is set to a light amount appropriate for the subject's eye E. More specifically, if the subject's eye E has a standard pupil diameter, the examiner can use an easily visible fixation target with a normal light amount. If the subject's eye E has a small pupil diameter, the examiner can use a miosis-free fixation target with a low light amount.

Now, if the switching of the eye refractive power measurement diaphragm for the subject's eye E having a small pupil diameter is late, the subject's eye E may sometimes produce miosis before the fixation target illumination light source 217 is set to the low light amount. As illustrated in FIG. 5B, in step S103a, the fixation target light amount control unit 300 may set the fixation target illumination light source 217 to the low light amount. In step S104a, the system control unit 401 switches to the small pupil diameter diaphragm 225.

As illustrated in FIG. 5C, step S103a where the fixation target light amount control unit 300 sets the fixation target illumination light source 217 to the low light amount and step S104a where the system control unit 401 switches to the small pupil diameter diaphragm 225 may be simultaneously executed. In such a case, the amount of light of the fixation target 216 is maintained or changed to a light amount smaller than that for a standard eye in an interlocked manner with the switching of the diaphragm apertures or a light shielding unit to be described below. Note that “simultaneously” refers to a concept that covers both simultaneously and generally simultaneously.

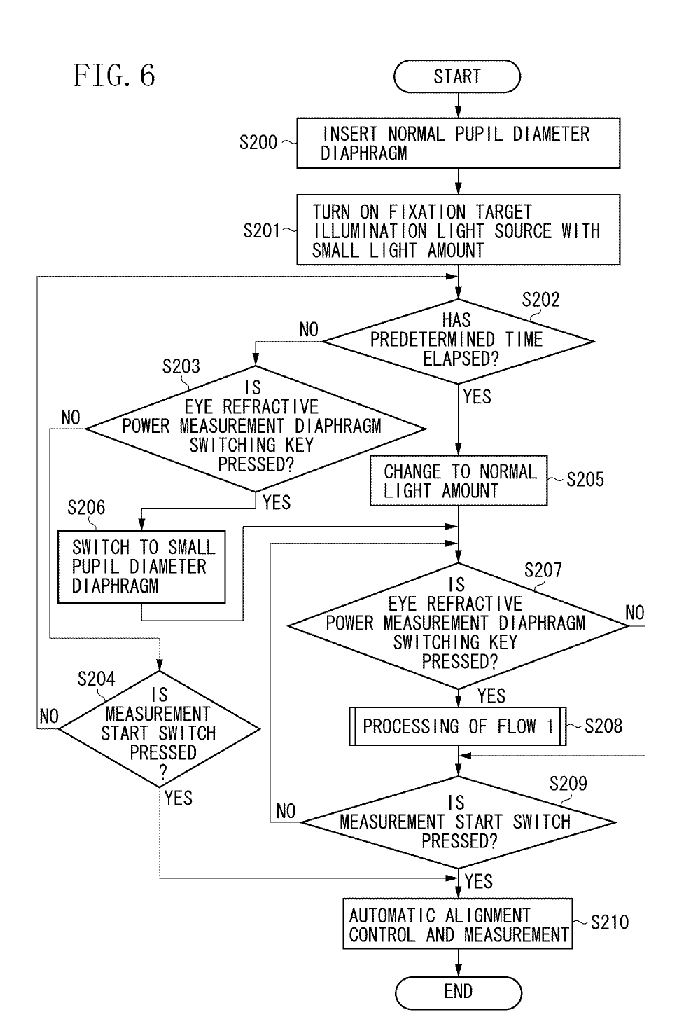

As illustrated in FIG. 6, the amount of light of the fixation target 216 may be maintained or changed to a light amount smaller than that for the standard eye before the switching of the diaphragm aperture regardless of the size of the diaphragm aperture. In such a case, processing for setting the fixation target illumination light source 217 to a low light amount is added at the start of test. In step S200 of FIG. 6, at the start of test, the system control unit 401 inserts the standard pupil diameter diaphragm 207, which is a normal pupil diameter diaphragm. In step S201, the fixation target illumination light source 217 is lit with a low light amount in advance. This prevents miosis at the start of test even if the pupil diameter of the subject's eye E is unknown.

As illustrated in step S202 of FIG. 6, processing may be added for determining whether a time (a predetermined time) needed to check the pupil diameter of the subject's eye E has elapsed and automatically changing the fixation target illumination light source 217 to a normal light amount after the lapse of the predetermined time. If the predetermined time has not yet elapsed (NO in step S202), the system control unit 401 proceeds to step S203. In step S203, if the examiner determines that processing on a subject's eye E having the small pupil diameter is needed, and presses the eye refractive power measurement diaphragm switching key 117 (YES instep S203), the system control unit 401 proceeds to step S206.

If the subject's eye E has the small pupil diameter and the eye refractive power measurement diaphragm switching key 117 is pressed in step S203, the diaphragm diameter and the amount of light of the fixation target 216 normally need to be changed. Since the amount of light of the fixation target 216 has already been changed (dimmed) in step S201, then in step S206, the system control unit 401 changes only the diaphragm diameter (switches to the small pupil diameter diaphragm. 225).

More specifically, when the eye refractive power measurement diaphragm is switched to the small pupil diameter diaphragm 225 by a switching unit, if the amount of light before the switching is a light amount smaller than a normal light amount (a predetermined light amount, the fixation target light amount control unit 300 maintains the amount of light of the fixation target 216). In step S207, the eye refractive power measurement diaphragm switching key 117 is not determined to be pressed (NO in step S207), and the system control unit 401 proceeds to step S209. In step S209, the system control unit 401 becomes ready for measurement.

Now, before the time (the predetermined time) needed to check the pupil diameter of the subject's eye E has elapsed, if the subject's eye E to be measured has the standard pupil diameter (NO in step S203), the system control unit 401 proceeds to step S204. In step S204, if the measurement start switch 404 is pressed (YES in step S204), then instep S210, the system control unit 401 immediately starts the automatic alignment control and becomes ready for measurement.

In FIG. 6, if the time (the predetermined time) needed to check the pupil diameter of the subject's eye E has elapsed (YES instep S202), then in step S205, the fixation target light amount control unit 300 changes the amount of light of the fixation target 216 to the normal light amount. After the lapse of the time (the predetermined time) required to check the pupil diameter of the subject's eye E, if the subject's eye E to be measured has the small pupil diameter, then in step S207, the eye refractive power measurement diaphragm switching key 117 is determined to be pressed (YES in step S207). The system control unit 401 proceeds to step S208.

In step S208, the system control unit 401 performs the processing of FIGS. 5A, 5B, and 5C (referred to as a flow 1, which includes steps S103 and S104 in FIG. 5A, or steps S103a and S104a in FIGS. 5B and 5C). The system control unit 401 proceeds to step S209. If the measurement start switch 404 is pressed (YES in step S209), then in step S210, the system control unit 401 starts the automatic alignment control and becomes ready for measurement.

As described above, according to the present exemplary embodiment, the small pupil diameter diaphragm 225 is automatically inserted into the optical path 02 and the amount of light emitted from the fixation target illumination light source 217 is reduced in response to the pressing of the eye refractive power measurement diaphragm switching key 117. The examiner can thus perform measurement of a subject having small pupils by a simple operation.

Since the small pupil diameter diaphragm 225 is automatically inserted into the optical path 02 and the amount of light emitted from the fixation target illumination light source 217 is reduced by a simple operation, the examiner can promptly perform measurement of a subject having small pupils. The prompt measurement can prevent miosis occurring from an increased period of measurement preparation.

According to the present exemplary embodiment, even if the standard pupil diameter diaphragm. 207 is inserted in the optical path 02, the amount of light emitted from the fixation target illumination light source 217 can be reduced for a predetermined time as with the case with the small pupil. This can prevent the subject's eye E from producing miosis while the examiner is determining the pupil diameter of the subject's eye E. The method for reducing the amount of light emitted from the fixation target illumination light source 217 for a predetermined time as with the case with the small pupil even if the standard pupil diameter diaphragm. 207 is inserted in the optical path 02 is effective to a subject who is known to have small pupils in advance.

In the present exemplary embodiment, the system control unit 401 can switch the light amount between two levels. However, the light amount need not be switched between two levels and may be switched among three levels or more. In exemplary embodiments where there are three or more types of eye refractive power measurement diaphragms corresponding to the pupil diameters of the subject's eye E and where the aperture area of an eye refractive power measurement diaphragm changes continuously, the amount of light of the fixation target illumination light source 217 can be changed in three levels or more or in a continuous manner.

In addition to the plurality of levels of the light amount, the fixation target illumination light source 217 may be turned off. In such an exemplary embodiment, the fixation target illumination light source 217 may be a turned-off state immediately after a test start, and an appropriate light amount corresponding to the pupil diameter of the subject's eye E can be set when the fixation target illumination light source 217 is turned on.

Step S201 for setting the fixation target illumination light source 217 to a low light amount at the start of test and step S205 for adjusting the fixation target illumination light source 217 to a normal light amount after the lapse of the predetermined time (step S202), which are described in the present exemplary embodiment, are not indispensable elements of an exemplary embodiment of the present invention. In an exemplary embodiment where the fixation target illumination light source 217 is set to a normal light amount at the start of test, the examiner may take care that the subject's eye E having the small pupil diameter does not produce miosis immediately after a test start.

The eye refractive power measurement diaphragm switching key 117 may be a different switching unit. For example, if an eye refractive power measurement apparatus has a plurality of measurement modes and uses different eye refractive power measurement diaphragms in the respective modes, a mode switching unit may also serve as a switching unit for switching the aperture area of the light shielding unit.

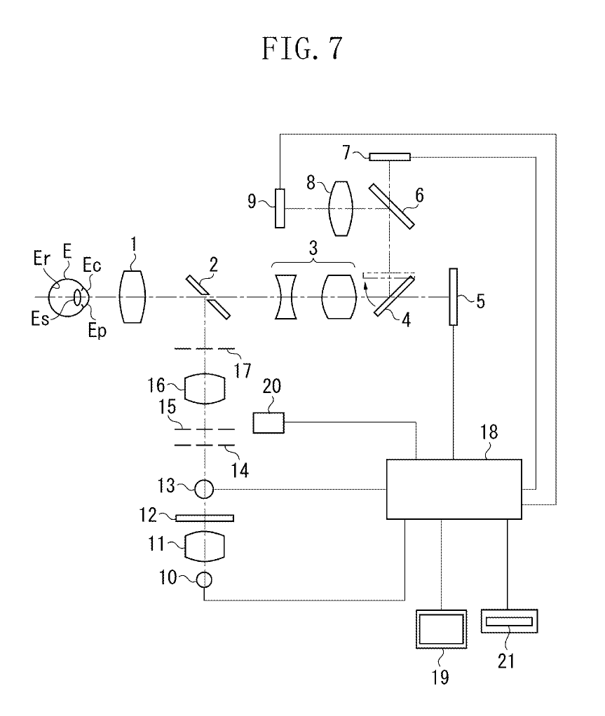

A second exemplary embodiment will be described below. The present exemplary embodiment is applied to an ophthalmologic imaging apparatus. FIG. 7 illustrates a block diagram of the ophthalmologic imaging apparatus. An objective lens 1 is arranged in front of a subject's eye E. A perforated mirror 2, photographic lenses 3, a movable mirror 4, and an imaging unit 5 are successively arranged on an optical path behind the objective lens 1 to constitute a fundus imaging optical system. The photographic lenses 3 can be moved for focusing. The imaging unit 5 includes a television camera having sensitivity to a visible wavelength range. A half mirror 6 and a fixation lamp 7 are arranged in a reflecting direction of the movable mirror 4. The fixation lamp 7 is located in a position generally conjugate with the fundus Er. A field lens 8 and a television camera 9 having sensitivity to an infrared wavelength range are successively arranged on a reflecting direction of the half mirror 6 to constitute an observation optical system.

An illumination optical system is arranged in an incident direction of illumination light on the perforated mirror 2. A condenser lens 11, a visible light cut filter 12, and a photographing light source 13 are successively arranged from the side of an observation light source 10 which emits visible light. An example of the observation light source 10 is a halogen lamp. The photographing light source 13 emits visible flash light. A ring slit 14, a crystalline lens baffle 15, and a relay lens 16 are also arranged in succession. The ring slit 14 has a ring-shaped opening and lies in a position generally optically conjugate with the pupil Ep of the subject's eye E. The crystalline lens baffle 15 includes light shielding portions or a ring-shaped opening (inner and outer light shielding portions forming a ring-shaped aperture therebetween), and is located in a position generally optically conjugate with the crystalline lens of the subject's eye E (for example, the posterior surface Es of the crystalline lens of the subject's eye E).

A cornea baffle 17 having a ring-shaped aperture is further arranged in a position generally optically conjugate with the cornea Ec of the subject's eye E. In FIG. 7, the outputs of the imaging unit 5 and the television camera 9 are connected to a control unit 18. The control unit 18 is connected with the fixation lamp 7, the observation light source 10, the photographing light source 13, a television monitor 19, a detection unit 20, and an image recording medium 21. The detection unit 20 detects a state of the crystalline lens baffle 15. An example of the detection unit 20 is a microswitch. In the first exemplary embodiment, the fixation target light amount control unit 300 controls the amount of light of the fixation target 216 according to switching of the size of the eye refractive power measurement diaphragm. In the present exemplary embodiment, the control unit 18 has such a function.

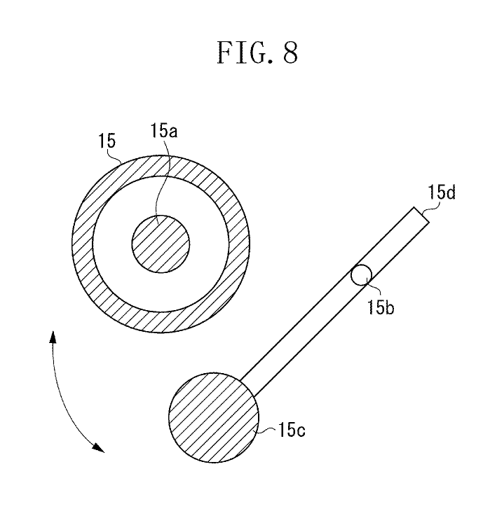

FIG. 8 illustrates a front view of the crystalline lens baffle 15. The crystalline lens baffle 15 includes a fixed light shielding portion 15a and a movable light shielding portion 15c. The light shielding portion 15a is located in the center and shields harmful light. The movable light shielding portion 15c is manually or electrically rotatable about a fulcrum 15b in the direction of the arrow and covers the light shielding portion 15a.

The movable light shielding portion 15c can be inserted into and removed from the optical path to change the area of a light shielding portion. The photographer checks whether a photographing range, position, and focusing are favorable, and then operates a not-illustrated photographing switch to perform still image photographing. Detecting the input of the photographing switch, the control unit 18 flips up the movable mirror 4 to retract the movable mirror 4 from the optical path and makes the photographing light source 13 emit light.

Like observation light, a photographing light flux emitted from the photographing light source 13 passes through the ring slit 14 and the ring-shaped opening of the crystalline lens baffle 15. The photographing light flux then passes through the relay lens 16 and the cornea baffle 17, and is reflected to the left by the peripheral mirror portion of the perforated mirror 2. The photographing light flux illuminates the fundus Er through the objective lens 1 and the pupil Ep of the subject's eye E. The reflected light of the illuminated fundus Er passes through the objective lens 1 and the hole portion of the perforated mirror 2 to form an image on an imaging surface of the imaging unit 5 through the photographic lenses 3. The control unit 18 displays the fundus image on the television monitor 19 and records the image on the image recording medium 21.

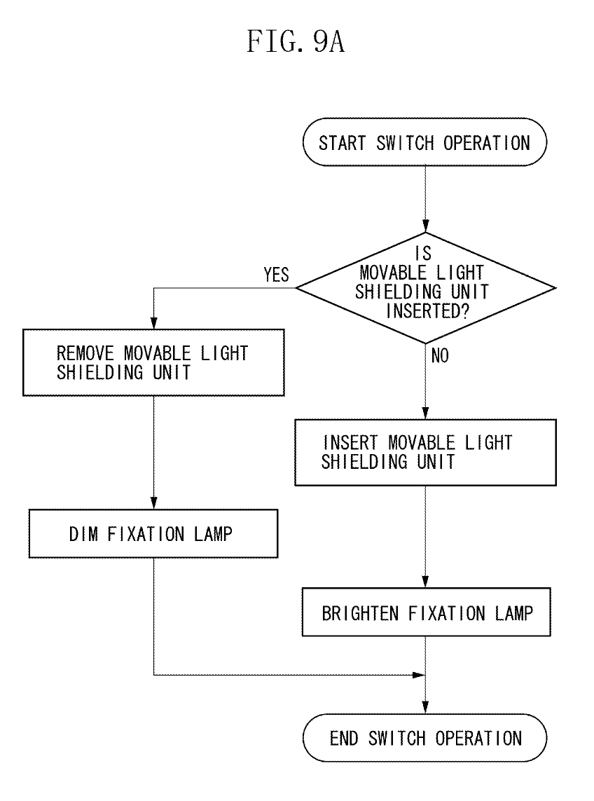

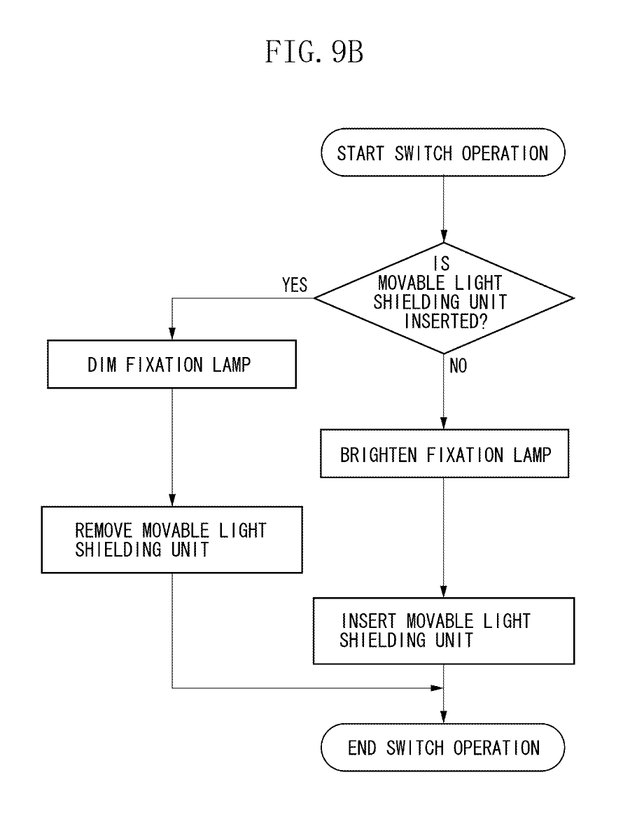

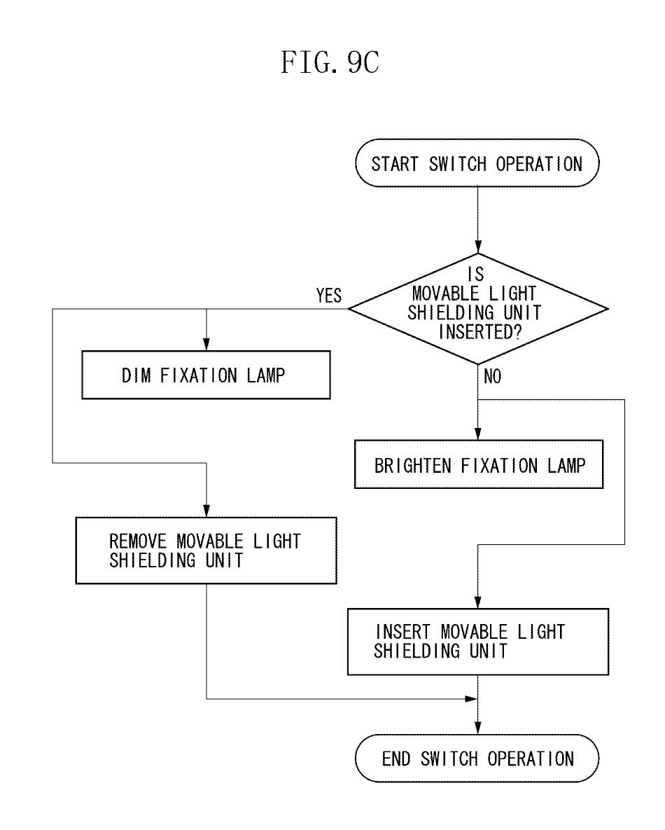

If the subject's eye E is insufficiently dilated and has a small pupil diameter, the captured fundus image is dim in the center. If the subject's eye E has a small pupil diameter, the photographer then presses a crystalline lens baffle insertion and removal switch (not illustrated) to retract the movable light shielding portion 15c of the crystalline lens baffle 15 illustrated in FIG. 8 from the optical path. Only the fixed light shielding portion 15a having a small light shielding area is placed on the optical axis to change the incident area of the light flux. FIGS. 9A, 9B, and 9C illustrate control when the crystalline lens baffle insertion and removal switch is pressed.

FIG. 9A is a flowchart when the amount of light of the fixation lamp 7 is maintained or changed to a light amount smaller than that for a standard eye after switching of the size of the light shielding portion of the crystalline lens baffle 15. FIG. 9B is a flowchart when the amount of light of the fixation lamp 7 is maintained or changed to the light amount smaller than that for the standard eye before the switching of the size of the light shielding portion of the crystalline lens baffle 15. FIG. 9C is a flowchart when the amount of light of the fixation lamp 7 is maintained or changed to a light amount smaller than that for the standard eye in an interlocked manner with the switching of the size of the light shielding portion of the crystalline lens baffle 15.

A description of FIGS. 9A to 9C is similar to that of FIGS. 5A to 5C if the insertion of the small pupil diameter diaphragm 225 into the optical path 02 according to the first exemplary embodiment is replaced with the removal of the movable light shielding portion 15c of the crystalline lens baffle 15, and the insertion of the standard pupil diameter diaphragm 207 into the optical path 02 is replaced with the insertion of the movable light shielding portion 15c.

In such a manner, the present exemplary embodiment can provide similar effects to those of the first exemplary embodiment.

In the present exemplary embodiment, the size of the opening of the crystalline lens baffle 15 is reduced and the amount of light of the fixation lamp 7 is reduced if the subject's eye E has a small pupil diameter. However, this is not restrictive. For example, the cornea baffle 17 optically conjugate with the cornea Ec of the subject's eye E may include a movable light shielding portion similar to the movable light shielding portion 15c. If the subject's eye E has a small pupil diameter, the size of the opening of the cornea baffle 17 may be reduced by using the movable light shielding portion of the cornea baffle 17 while the amount of light of the fixation lamp 7 is reduced.

If the subject's eye E has a small pupil diameter, the opening of the crystalline lens baffle 15 and the opening of the cornea baffle 17 both may be reduced in size while the amount of light of the fixation lamp 7 is reduced. The crystalline lens baffle 15 and the cornea baffle 17 correspond to a light flux limiting unit for limiting incidence of a light flux on the subject's eye E.

The opening of the crystalline lens baffle 15 and the opening of the ring slit 14 may be reduced in size.

The opening of the ring slit 14 may be reduced in size by providing a member corresponding to the movable light shielding portion 15c or by using a plurality of ring slits having openings of respective different sizes. The crystalline lens baffle 15 may also include a plurality of crystalline lens baffles having openings of respective different sizes to switch. The cornea baffle 17 may include a plurality of cornea baffles having opening of respective different sizes to switch. The technical items disclosed in the foregoing exemplary embodiments may be combined and/or modified as appropriate without departing from the scope of the exemplary embodiments of the present invention. A modification is described below.

In the foregoing exemplary embodiments, the switching unit for switching the size of the diaphragm aperture or the light shielding unit (the baffle) and the control unit for controlling the amount of light of the fixation target 216 or the fixation lamp 7 are described to be included in the main body of the ophthalmologic apparatus or the ophthalmologic imaging apparatus. However, an exemplary embodiment of the present invention is not limited thereto. The fixation target 216 or the fixation lamp 7 may be included in the main body of the ophthalmologic apparatus or the ophthalmologic imaging apparatus while the switching unit for switching the size of the diaphragm aperture or the light shielding unit (the baffle) and the control unit for controlling the amount of light of the fixation target 216 or the fixation lamp 7 may be provided as an ophthalmologic control apparatus outside the main body of the ophthalmologic apparatus or the ophthalmologic imaging apparatus.

Aspects of the present invention can also be realized by a computer of a system or apparatus (or devices such as a CPU or MPU) that reads out and executes a program recorded on a memory device to perform the functions of the above-described embodiment (s), and by a method, the steps of which are performed by a computer of a system or apparatus by, for example, reading out and executing a program recorded on a memory device to perform the functions of the above-described embodiment (s). For this purpose, the program is provided to the computer for example via a network or from a recording medium of various types serving as the memory device (e.g., computer-readable medium).

While the present invention has been described with reference to exemplary embodiments, it is to be understood that the invention is not limited to the disclosed exemplary embodiments. The scope of the following claims is to be accorded the broadest interpretation so as to encompass all modifications, equivalent structures, and functions.

This application claims priority from Japanese Patent Application No. 2012-030665 filed Feb. 15, 2012, which is hereby incorporated by reference herein in its entirety.

What is claimed is:

1. An ophthalmologic apparatus comprising:
a first changing unit configured to change a size of an aperture of a diaphragm arranged in an optical path connecting a subject's eye and a light source and in a position conjugate with a pupil of the subject's eye; and
a second changing unit configured to, if a signal for instructing the first changing unit to change the size of the aperture from a first size to a second size smaller than the first size is output to the first changing unit, change an amount of light of a fixation target image projected onto the subject's eye from a first light amount to a second light amount smaller than the first light amount.

2. The ophthalmologic apparatus according to claim 1, further comprising a signal output unit configured to output the signal for instructing the first changing unit to change the size of the aperture from the first size to the second size according to an instruction from an examiner.

3. The ophthalmologic apparatus according to claim 1, wherein the second changing unit is configured to change the amount of light of the fixation target image to the second light amount at the same time that the first changing unit changes the size of the aperture from the first size to the second size.

4. The ophthalmologic apparatus according to claim 1, wherein the second changing unit is configured to change the amount of light of the fixation target image to the second light amount before the first changing unit changes the size of the aperture from the first size to the second size.

5. The ophthalmologic apparatus according to claim 1, wherein the amount of light of the fixation target image is set to the second light amount before the signal for instructing the first changing unit to change the size of the aperture from the first size to the second size is output, and wherein the second changing unit is configured to maintain the second light amount if the signal for instructing the first changing unit to change the size of the aperture from the first size to the second size is output.

6. The ophthalmologic apparatus according to claim 5, wherein the second changing unit is configured to, if the signal for instructing the first changing unit to change the size of the aperture from the first size to the second size is not output to the first changing unit for a predetermined time, change the amount of light of the fixation target image from the second light amount to the first light amount.

7. The ophthalmologic apparatus according to claim 1, further comprising:
a first diaphragm having an aperture of the first size; and
a second diaphragm having an aperture of the second size,
wherein the first changing unit is configured to change the size of the aperture of the diaphragm arranged in the position conjugate with the pupil of the subject's eye by selectively inserting one of the first diaphragm and the second diaphragm into the optical path.

8. The ophthalmologic apparatus according to claim 1, further comprising a measurement unit configured to measure refractive power of the subject's eye based on a return beam of beams that are emitted from the light source and with which the subject's eye is irradiated through the aperture.

9. The ophthalmologic apparatus according to claim 2, further comprising a pressable switch,
wherein the signal output unit is configured to output the signal for instructing the first changing unit to change the size of the aperture from the first size to the second size according to pressing of the switch by the examiner.

10. The ophthalmologic apparatus according to claim 1, wherein each of the aperture of the first size and the aperture of the second size includes an annular aperture, and
wherein the aperture of the first size has a diameter greater than that of the aperture of the second size.

11. A control method comprising:
changing a size of an aperture of a diaphragm arranged in an optical path connecting a subject's eye and a light source and in a position conjugate with a pupil of the subject's eye; and
changing an amount of light of a fixation target image projected onto the subject's eye from a first light amount to a second light amount smaller than the first light amount,
wherein, if a signal for instructing changing of the size of the aperture from a first size to a second size smaller than the first size is output, the changing of the size of the aperture of the diaphragm and the changing of the amount of light of the fixation target image are executed.

12. A non-transitory computer-readable storage medium storing a program that causes a computer to execute the control method according to claim 11.
